(19) United States
(12) Patent Application Publication
(10) Pub. No.: US 2026/0096892 A1
(43) Pub. Date: Apr. 9, 2026

(54) MANDIBULAR AND CRANIAL PROSTHESIS FOR A TEMPOROMANDIBULAR JOINT, MANDIBULAR AND CRANIAL CUTTING GUIDE, TEMPOROMANDIBULAR JOINT AND SET, AND PROCEDURE FOR INSERTING A TEMPOROMANDIBULAR JOINT

(71) Applicant: INSTITUTO TECNOLÓGICO DE CANARIAS, S.A., Las Palmas De Gran Canaria (ES)

(72) Inventor: Donato Monopoli, Las Palmas De Gran Canaria (ES)

(21) Appl. No.: 19/114,301

(22) PCT Filed: Sep. 19, 2023
(86) PCT No.: PCT/EP2023/075852

§ 371 Date: Mar. 21, 2025

(30) Foreign Application Priority Data
Sep. 22, 2022 (DE) 10 2022 124 434.2

(51) Int. Cl. A61F 2/30; A61B 17/17; A61F 2/28

(52) U.S. Cl. CPC A61F 2/3099; A61B 17/1739; A61B 17/176; A61F 2/2803; A61F 2/30749; A61F 2002/2882; A61F 2002/30214; A61F 2002/30991

(57) ABSTRACT

Mandibular prosthesis (10) for a mandibular side of a temporomandibular joint, mandibular (200) and cranial cutting guide, cranial prosthesis (50) for the temporomandibular joint, temporomandibular joint (100) and set, and procedure for inserting a temporomandibular joint (100).

The invention relates to a mandibular prosthesis for a mandibular side of a temporomandibular joint and a cranial prosthesis for a cranial side of a tempomandibular joint to form and replace a temporomandibular joint. The invention also relates to a mandibular cutting guide and a cranial cutting guide, as well as a set of cutting guides and/or prostheses and a procedure for inserting a temporomandibular joint.

In a temporomandibular joint, also called a jaw joint, and the area around it, the consecutive area of the mandible (mandibula) and the temporal bone (os temporale) can be distinguished. In particular as regards the mandible, the condylar process, having a neck and (condylar) head (caput mandibulae) and as regards the temporal bone, the glenoid cavity (fossa glenoidalis), formed behind the zygomatic arc (arcus zygomaticus), including the muscle tissues and interstitial tissues connecting them to each other and the articular disc (discus articularis).

Patients suffering considerable damage to the temporomandibular joint resulting from accidents, ankylosis, rheumatoid arthritis, osteoarthritis and tumors often have problems chewing and speech problems. Further problems can be pain and opening restrictions.

Overcoming these problems can require partial, one sided full replacement or even both side replacement of the temporomandibular joints.

From U.S. Pat. No. 3,178,728 a one-sided prosthesis is known. It is formed by a chrome-cobalt casting for covering the glenoid cavity, including the anterior articular tubercle (tuberculum articulare), in order to provide a bearing for the condylar head. A comparable, yet shorter one-sided prosthesis is known from U.S. Pat. No. 3,579,643. This prosthesis leaves the glenoid cavity free. A more recent suggestion is described in US 2009/0222102.

Two-sided prostheses are known among others from U.S. Pat. Nos. 4,693,722, 4,778,472, 4,917,701, 5,549,680, 5,989,292 and French patent application FR 2,558,721.

A more recent design of a two-sided prosthesis is the subject of U.S. Pat. No. 6,132,466, comprising a replacement of the condylar head and a bearing for it. The condylar head prosthesis comprises a downwardly extending strip to be attached by means of screws to and below the neck of the condylar process. A comparable design is the subject of US 2009/0138092.

Another two-sided prosthesis is known from WO 2014/023903. The portion that is to replace the condylar head is two-part, having a slightly curved pin including prosthetic condylar head, which is to be attached in the jaw, bone. A retainer part comprising a downwardly extending strip to be attached by means of screws to and below the neck of the condylar process and a retaining ring integrally formed therewith for keeping the pin in the correct orientation.

US 2019/0192302 Al discloses a prosthesis for the mandibular side of the temporomandibular joint.

Comprising a head part to replace the condylar head at the anatomic location, and an attachment plate for attachment to the mandible, wherein the attachment plate has been formed having a lateral portion that has been formed and is intended for abutting the lateral side of the ascending branch of the mandible.

CN 210 749 669 U shows a custom-made condylar prosthesis.

BR 10 2019 025 804 A2 discloses a condylar prosthesis stabilised mainly by a support in the mandible and comprising a prosthesis for the joint responsible for the movement of the mandible in relation to the rest of the skull.

GB 248 06 82 A discloses various condylar prostheses and a cutting guide to remove the bone in the condylar region for a prosthesis.

A lockable temporomandibular joint prosthesis for replacing and correcting the temporomandibular joints of people with severe functional disorders, comprising temporal elements, a condylar plate and a condylar head is known from BR 20 2013 004 862 Y1.

EP 0 337 749 A1 shows a condyle mini prosthesis for attachment to the mandibular bone of a patient, said prosthesis having a condylar head defining a superior articular convexo-planar surface for substantially planar load bearing contact with a correspondingly concavo-planar surface of a prosthetic glenoid fossa.

The condylar head has a substantially planar surface inferior to the superior surface and which, when the prosthesis is in position, abuts a substantially planar superior cut surface provided on the natural condyle.

U.S. Pat. No. 9,517,135 B2 discloses a condylar prosthesis for a temporo-mandibular joint. Which includes a mandibular implant, which is intended to be rigidly attached to the branch of a mandible, and a joint head, which is intended to serve as a replacement mandibular condyle, the mandibular implant including an anchoring stem permitting the use of an intramedullary connection in the summit of the branch of the mandible, and the distal end of the anchoring stem being offset towards the front of the prosthesis with respect to the joint head and in a median plane of the prosthesis. The anchoring stem follows a curved profile along its length and in the median plane of the prosthesis, the direction of curvature of the anchoring stem being oriented towards the front of the prosthesis.

The objective of the invention is to increase the safety of the patient during and after replacing a temporomandibular joint and to restore a physiologic function compared to the previous art.

The objective of the invention is achieved by providing a mandibular prosthesis for a mandibular side of a temporomandibular joint. The mandibular prosthesis comprises a head part to replace the condylar head, in particular at the anatomic location and an attachment plate for attachment to the mandible. The attachment plate comprises a lateral portion that has been formed and is intended for abutting the lateral side of the ramus and an upper portion, forming one unity with the lateral portion. The upper portion comprises at its underside a plane support surface for resting on a flat cutting surface provided at the upper side of the ramus after removing the condylar head. The head part of the mandibular prosthesis comprises a mounting section for mounting a joint head, wherein the mounting section is forming one unity with upper portion. By making the mandibular prosthesis in two or more parts further customization options can be realized. The joint head is a vital part of the temporomandibular joint, facilitating the joint movement as it interacts with most of the further components directly. Forming a defined unity with the mounting section of the mandibular prosthesis is important to a safe and reliable function of the prosthesis. The joint head can be manufactured of polymeric materials like UHMWPE (Ultra High Molecular Weight Polyethylene) or PEEK (Polyether ether ketone) material and could be provided in various sizes and dimensions to fit the needs of the patient.

Temporomandibular joints are often repaired or replaced when the condylar head is damaged due to an accident or due to malformation of the bones, surrounding tissue, tendon or muscles. Usually the temporomandibular joint needs to withstand high loads while being a relative small joint. Also mobility in all directions has to be ensured to provide the patient with a working joint. Alignment of the mandible to the maxilla is important in repairing the temporomandibular joint. With a mandibular prosthesis according to the invention an exact alignment is easily possible and all needs are beneficially facilitated. Especially the plane support surface at the underside of the upper portion enables an alignment and therefore a reliable function and a beneficial force transmission from the prosthesis into the mandible. The corresponding and parallel surfaces in form of a cutting surface specifically cut to the support surface of the prosthesis by removing the condylar head and the support surface make the positioning of the prosthesis a very safe procedure with ensured success. Further the support means on a lateral side and a medial side facilitate further support and force transmission when loads from different directions are applied to the joint. A mandibular prosthesis exhibiting the said characteristics can also be smaller than the previous art. The invention minimizes the surgical approaches, reduces bone sacrifice as less cutting of bone is required, restores the anatomical rotation center of the joint and preserves soft tissues surrounding the joint. Due to limited dissection in the temporomandibular joint communication with oral cavity is avoided when a simultaneous orthognatic surgery is performed. A smaller prosthesis does not require large incisions in the skin for implantation, instead only a small incision is necessary in the region of the auditory canal. With less damage to the surrounding tissue and a perfectly defined positioning of the prosthesis the replacement of the temporomandibular joint with a prosthesis according to the invention is resulting in a safer and better process for the patient also after the implant in the healing, functional movements and rehabilitation.

Further, with a prosthesis according to the invention, the reproducibility of the results in terms of positioning the prosthesis, the force induction and safety is ensured. While the previous art the exact positioning of the prostheses on the mandible was not ensured.

The mandibular prosthesis is preferably manufactured from Ti6Al4V powder, in particular it is custom made according to 3D scans of the patients anatomical features for whom it is intended. Titanium has properties that favor osseointegration and is bio compatible thus making the implant especially safe component for temporomandibular joint replacement.

It is beneficial if the mandibular prosthesis comprises a medial portion that is intended for abutting the medial side of the ramus and forming one unity with the upper portion.

It is conceivable that the mounting section is cylinder-shaped or cone-shaped. This shape can aid to position the joint head onto the prosthesis in a defined way.

Advantageously the joint head and the mounting section are configured such that the joint head is clamped onto the mounting section. This could be achieved by providing the mounting section as a morse cone and by providing a hollow morse cone in a mounting section opening in the joint head as corresponding sections. Morse cones are a beneficial way of connecting two components with no or very little tools required, also a morse cone mounting enables the mounting in any rotational position around an axis of the morse cone mounting.

The lateral portion could comprise holes arranged in a hole pattern for attachment to the lateral side of the ramus. The mandibular prosthesis needs to be fixed to the mandible in a defined position to avoid shifting and enable the osseointegration process after inserting. Preferably the mounting holes have a thread or are configured in such a way that a thread can be formed so that screws with a threaded locking head can be used which secure the prosthesis even further. With the prosthesis secured in that way, shifting can be eliminated. Further, it is conceivable that the holes in the mandibular prosthesis are inclined in such a way that the holes are mostly not perpendicular to the bone surface, but are inclined towards the incision in the skin. The inclination of the holes can vary and can be different for all holes, this increases the stability of the fixation of the prosthesis to the mandible and enables a safe way to introduce and fasten the screws.

The objective of the invention is also achieved by a mandibular cutting guide. In particular, the mandibular cutting guide is for arrangement at the ramus and at least partially around the condylar process. It comprises at least a lateral wall section for abutment against the outside of the ramus and a guide surface for guiding a cutting instrument when cutting off the condylar head along the guide surface. The guide surface is preferably planar; this enables the creation and preparation of a surface for optimal interaction with the underside of the mandibular prosthesis. It is conceivable, that not only the condylar head can be cut off with the cutting guide, but also the condylar process further in rostral direction. The cutting guide further enables a beneficial cutting of the mandible bone in a defined plane along the guiding surface. With a desired cutting surface a good positioning of the mandibular prosthesis on the ramus is ensured thus improving the safety of the patient and it allows to set the normal occlusion.

It is beneficial, if the mandibular cutting guide comprises a medial wall section for abutment against the inside of the ramus. It is also conceivable, that the mandibular cutting guide further comprises an anterior and/or a posterior wall section for abutment against the anterior and/or posterior side of the ramus. Through the different medial, anterior and/or posterior wall sections, the mandibular cutting guide can be positioned safely on the mandible, which makes the cutting process more precise and safer.

Preferably, the mandibular cutting guide is being formed of an elastically deformable material that can be bent to be placed around the condylar process. Through the deformable material the cutting guide can be beneficially placed through a small incision onto the mandible and does not need as much space as a rigid cutting guide, additionally the surrounding tissue will be less stressed. Further also the removal after the cutting is easily facilitated.

It is conceivable that the mandibular cutting guide comprises holes arranged in a hole pattern for attachment to the lateral side of the ramus. Through the holes the cutting guide can be attached safely to the mandible with screws after being positioned. Through a safe attachment, further shifting of the cutting guide can be avoided, for instance while cutting. Cutting could apply forces to the cutting guide and cause a move in the position on the mandible which would lead to a wrong cut.

The guide surface of the mandibular cutting guide provides a cutting-start-region and a cutting-end-region, wherein a cutting stopper is provided at the distal end of the cutting-end-region. This aids for a more precise cut and avoids slipping into surrounding tissue with the tool used for cutting. Further the cutting stopper could provide a slot that encloses the cutting-end region from three sides. A more defined end of the cutting movement is achieved through the enclosement of the cutting end region, with no slipping or sliding into the surrounding tissue at the cutting region with the tool used for cutting the condylar region.

The objective of the invention is also achieved by a cranial cutting guide. In particular the cranial cutting guide is for arrangement at the zygomatic arc in the region of the temporomandibular joint and comprises at least a lateral wall section for abutment against the outside of the zygomatic arc and a planar guide surface for guiding a cutting instrument when cutting off parts of the zygomatic arc. The guide surface is preferably planar; this enables the creation and preparation of a surface for optimal interaction with the cranial prosthesis.

The guide surface of the cranial cutting guide provides a cutting-start-region and a cutting-end-region, and wherein a cutting stopper is provided at the distal end of the cutting-end-region. This aids for a more precise cut and avoid slipping into surrounding tissue with the tool used for cutting. Further the cutting stopper could provide a slot that encloses the cutting-end region from three sides. A more defined end of the cutting movement is achieved of the cutting end region, with no slipping or sliding into the surrounding tissue at the cutting region with the tool used for cutting on the zygomatic arch region.

The objective of the invention is also achieved by a one piece cutting guide. The one piece cutting guide comprises a first part with a mandibular cutting guide and a second part comprising a cranial cutting guide. Through a combination of two cutting guides into a single piece the cutting is beneficially improved. In particular the cranial cutting guide is for arrangement at the zygomatic arc in the region of the temporomandibular joint and comprises at least a lateral wall section for abutment against the outside of the zygomatic arc and a planar guide surface for guiding a cutting instrument when cutting off parts of the zygomatic arc. In particular, the mandibular cutting guide is for arrangement at the ramus and at least partially around the condylar process. The mandibular cutting guide comprises at least a lateral wall section for abutment against the outside of the ramus and a guide surface for guiding a cutting instrument when cutting off the condylar head along the guide surface.

The objective of the invention is also achieved by providing a cranial prosthesis for a cranial side of a temporomandibular joint. The cranial prosthesis comprises a lateral portion that has been formed and is intended for abutting the lateral side of zygomatic bone of the cranium, a main body, forming one unity with the lateral portion, comprising an upper portion that has been formed and is intended for abutting the fossa.

The cranial prosthesis is preferably manufactured from Ti6Al4V powder, in particular it is custom made according to 3D scans of the patient's anatomical features for whom it is intended. Titanium has properties that favor osseointegration and is biocompatible thus making the implant especially a safe component for temporomandibular joint replacement.

It is conceivable for the cranial prosthesis that a porous structure is provided at the upper side of upper portion intended to be osseointegrated into the cranium. The porous structure could be a grid like structure to improve the osseointegration process after the insertion of the prosthesis.

The lateral portion of the cranial prosthesis could comprise holes arranged in a hole pattern for attachment to the lateral side of the zygomatic bone of the cranium. The cranial prosthesis needs to be fixed to the cranium in a defined position to avoid shifting and enable the osseointegration process after inserting. Preferably the mounting holes have a thread or are configured in such a way that a thread can be formed so that screws with a threaded locking head can be used which secure the prosthesis even further. With the prosthesis secured in that way, shifting can be eliminated. Further, it is conceivable that the holes in the cranial prosthesis are inclined in such a way that the holes are mostly not perpendicular to the bone surface, but are inclined towards the incision in the skin. The inclination of the holes can vary and can be different for all holes, this increases the stability of the fixation to the zygomatic bone and the fossa and enables a safe way to introduce and fasten the screws.

The objective of the invention is also achieved by providing a temporomandibular joint. The temporomandibular comprises a mandibular prosthesis and a cranial prosthesis according to the invention. Through the combination of the two prostheses that are corresponding to each other a better and safer result for the patient can be achieved.

The objective of the invention is also achieved by a set comprising:
a mandibular prosthesis according to the invention,
a mandibular cutting guide according to the invention, and
joint heads of different sizes and/or with different mounting section openings to be mounted on the mounting section of the head part of the mandibular prosthesis.

It is conceivable, that the set also comprises a hammer for fixing the joint head with mounting section of the head part by a hammer stroke. A set like this provides the minimum required components for a replacement of the temporomandibular joint on one side.

It is beneficial, if the hole pattern of the lateral portion of the mandibular prosthesis is different than the hole pattern of the lateral wall section of the mandibular cutting guide. Different hole patterns are beneficial as it prevents failure of the attachment of the prostheses to the mandible as holes in the bone where already screws were inserted before could be weakened. Weak attachment could result in failure of the bone due to high forces in the joint.

It is further conceivable that the set comprises a cranial prosthesis according to the invention. It is beneficial if the set comprises a cranial cutting guide according to the invention and/or a one piece cutting guide according to the invention to provide a complete set for a temporomandibular joint replacement.

The objective of the invention is also achieved by a procedure for inserting a temporomandibular joint, comprising in particular a mandibular prosthesis according to the invention and a cranial prosthesis according to the invention and in particular by usage of the set according to the invention. The procedure comprises advantageously the following steps:
(a) providing a single access through a skin incision located in the area of the tragus in front of the auditory canal, or behind the auditory canal,
(b) removal of the periosteum on the zygomatic arch in the area where the cranial prosthesis is to be abutted,
(c) removal of the periosteum on the ramus in the area of the condylar process, where the mandibular cutting guide is to be abutted,
(d) positioning the mandibular cutting guide through the single access so that it abuts the mandibular notch and the medial part of the ramus and fasten the mandibular cutting guide with
(e) cutting off the condylar process through the single access with a cutting instrument along the guide surface of the mandibular cutting guide to provide a flat cutting surface at the ramus,
(f) removing the screws and the mandibular cutting guide,
(g) positioning the cranial prosthesis through the access so that its lateral portion abuts the lateral side of zygomatic bone and its upper portion abuts the fossa,
(h) fastening the cranial prosthesis with screws through the single access,
(i) placing a joint head on the mounting section of the mandibular prosthesis,
(j) positioning the mandibular prosthesis through the single access so that its lateral portion abuts the lateral side of the ramus and its support surface rests on the cutting surface of the ramus,
(k) verifying the correct match and placement between the joint head of the mandibular prosthesis and the joint pan of the cranial prosthesis,
(l) fastening the mandibular prosthesis with screws through the single access.

It is beneficial, if the verifying according to step (k) is done in case that there is an incorrect match or placement due to too large or too small clearance by removing the joint head and placing and fixing a smaller or larger joint head on the mounting section of the It is conceivable, that the fixing of the joint head on the mounting section is done by a hammer impact.

Preferably, the fastening of the cranial prosthesis with screws according to step (h) is done such that first the most posterior screws are fastened. This is beneficial, because their orientation in a cranial direction favors the implants porous structure to come into contact with the fossa.

Further it is conceivable, that the screws provided for fastening the cranial prosthesis and the mandibular prosthesis have a diameter from 1 mm to 4 mm, preferably of 2 mm and 3 mm have a length from 4 mm to 14 mm, preferably from 7 mm to 9 mm. Through providing screws within these dimensions, the fastening can be achieved to withstand the high loads that can occur in the joint and applied to the prostheses.

Further details and advantageous embodiments of the invention can be found in the following description, by means of which an embodiment of the invention is described and explained in more detail.

### SHOWING:

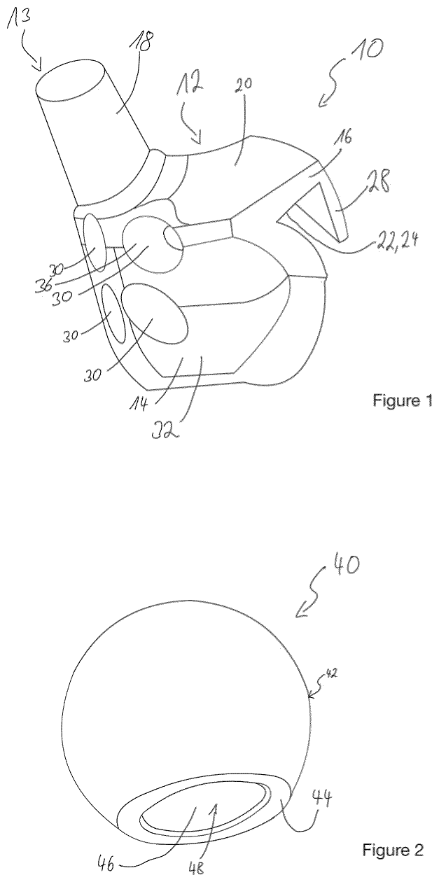

FIG. 1: A mandibular prosthesis;

FIG. 2: A joint head;

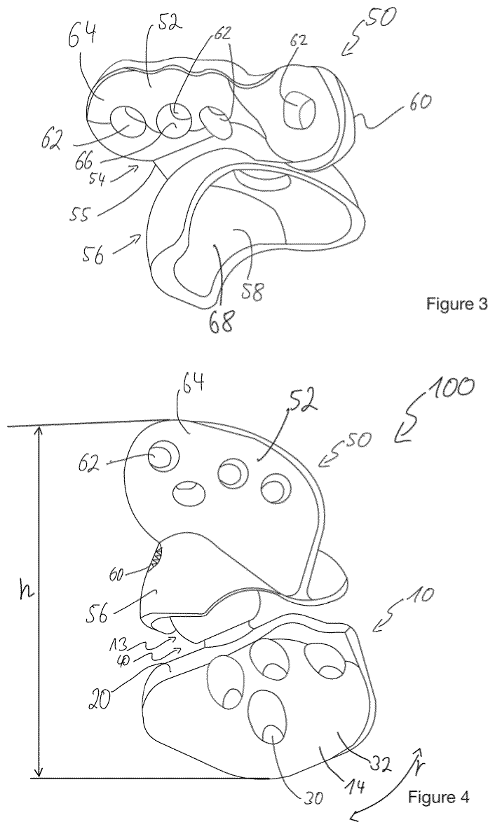

FIG. 3: A cranial prosthesis;

FIG. 4: A temporomandibular joint prosthesis

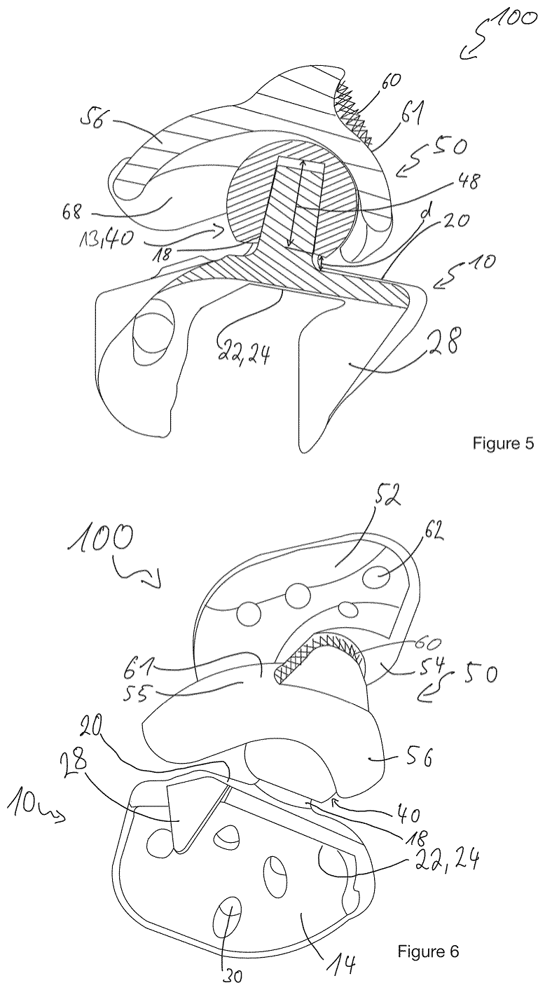

FIG. 5: A cut view through the temporomandibular joint prosthesis of FIG. 4;

FIG. 6: The temporomandibular joint prosthesis of FIG. 4 from a medial position;

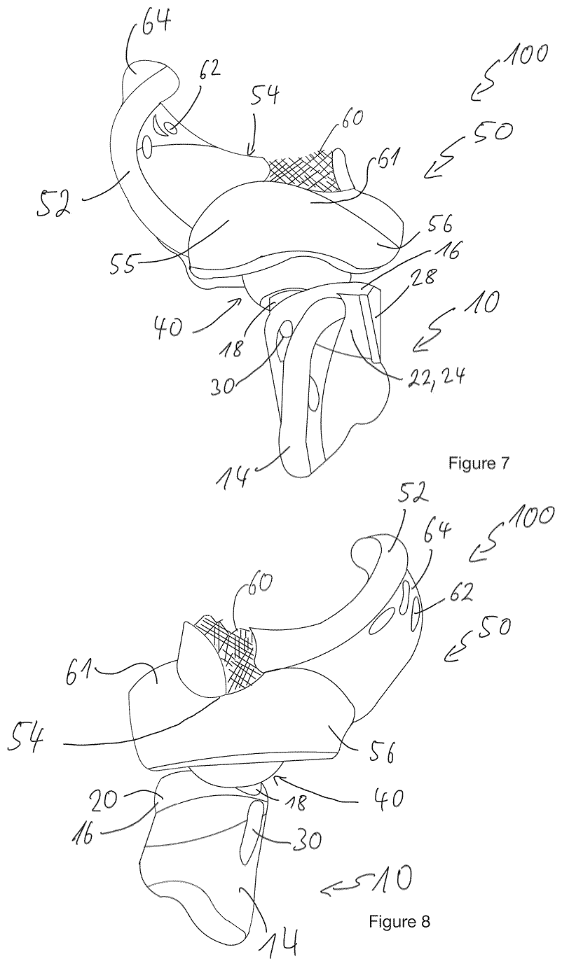

FIG. 7: The temporomandibular joint prosthesis of FIG. 4 from a rostral position;

FIG. 8: The temporomandibular joint prosthesis of FIG. 4 from an occipital position;

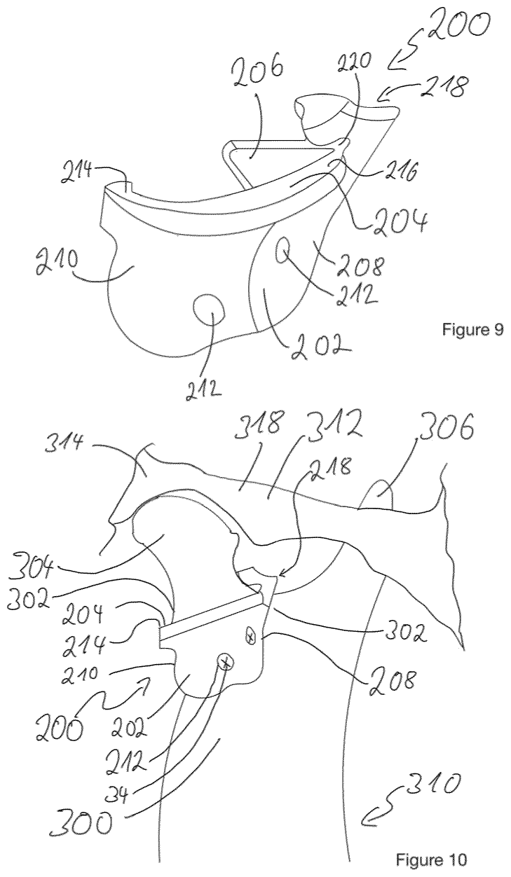

FIG. 9: a Mandibular Cutting Guide;

FIGS. 10 to 12: The mandibular cutting guide of FIG. 9 arranged at the condylar process;

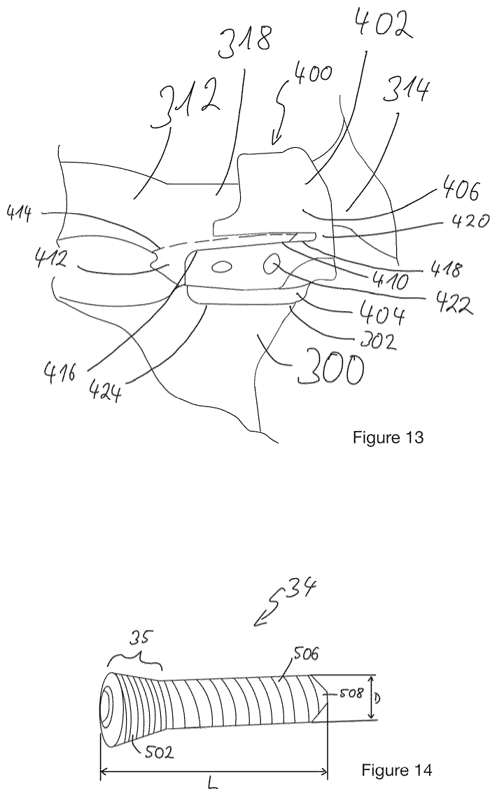

FIG. 13: A one piece cutting guide arranged at the temporomandibular joint;

FIG. 14: a Screw;

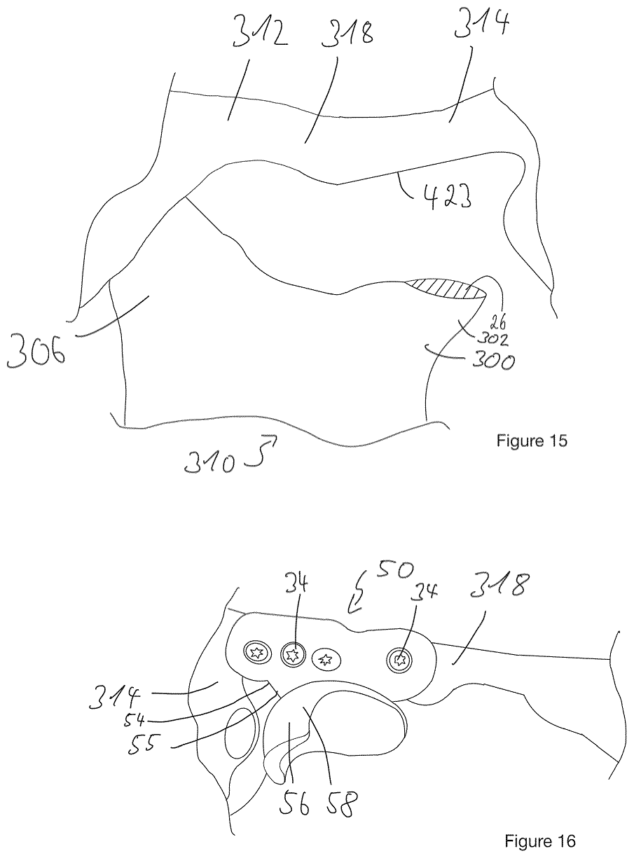

FIG. 15: A cutting surface on the mandible;

FIG. 16: The cranial prosthesis arranged at the zygomatic arch;

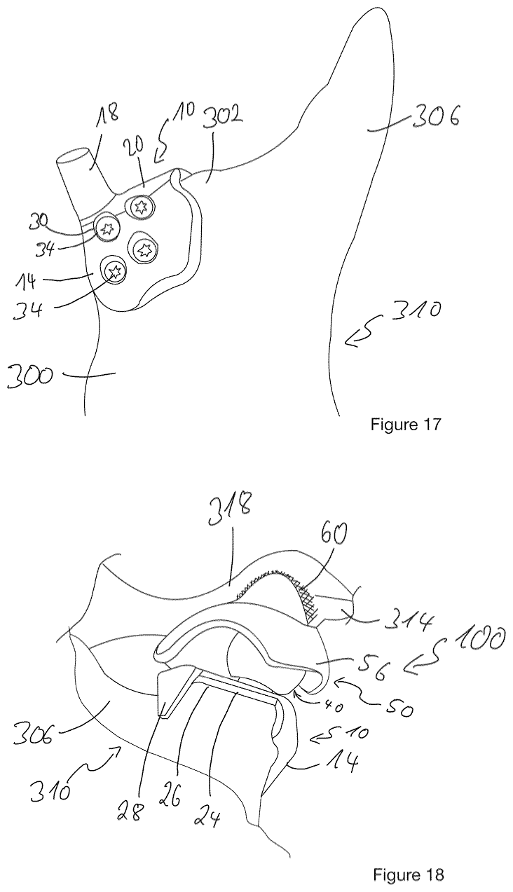

FIG. 17: The mandibular prosthesis arranged at cutting surface on the mandible;

FIG. 18: The temporomandibular joint prosthesis inserted with the cutting surface on the mandible;

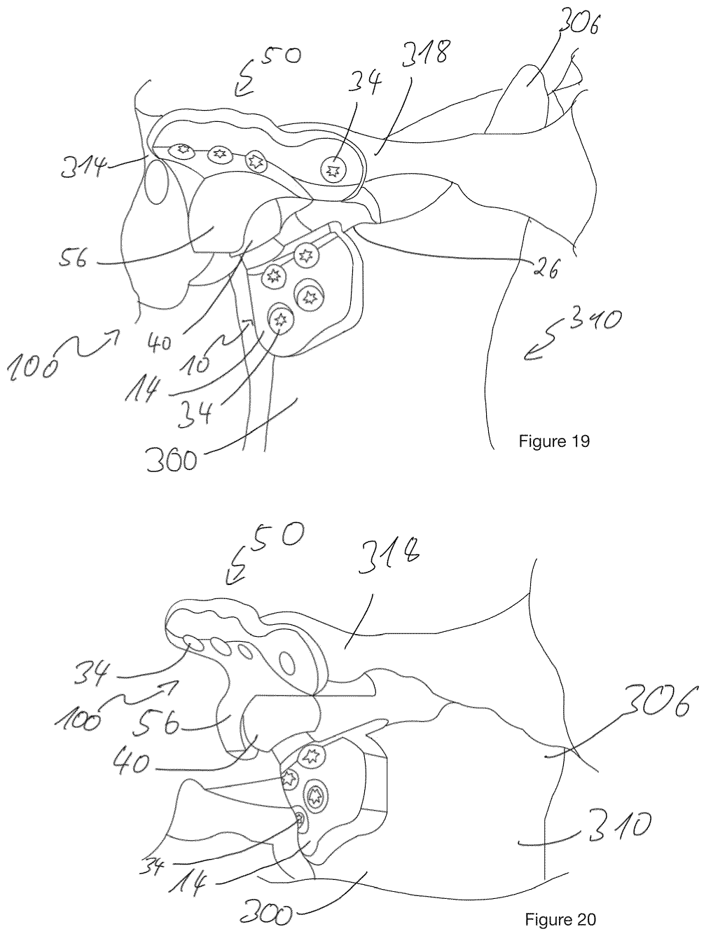

FIG. 19: The temporomandibular joint prosthesis inserted from a temporal position;

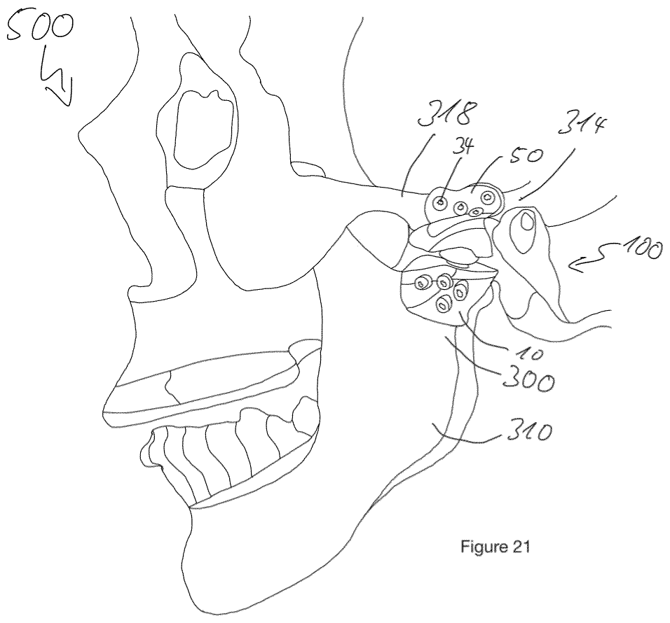

FIG. 20: The temporomandibular joint prosthesis inserted from a occipital position;

FIG. 21: The temporomandibular joint prosthesis inserted with a view of the skull and surrounding jaw bones; and

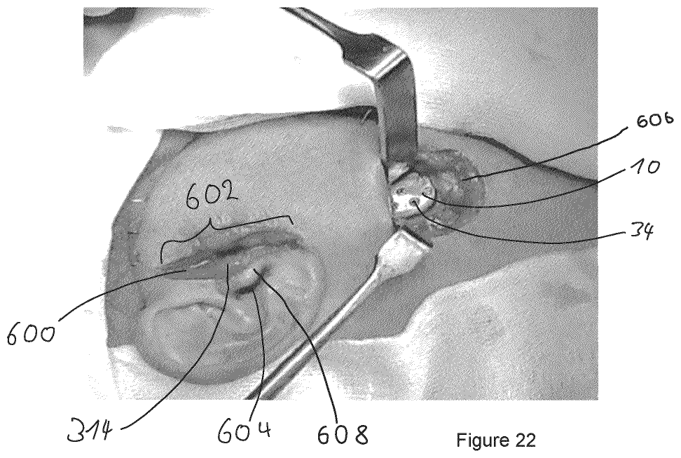

FIG. 22: A view of the incision in the skin necessary to insert the prostheses.

FIG. 1 shows a mandibular prosthesis 10 with an attachment plate 12 to the mandible. This embodiment is intended for the right side of the temporomandibular joint and is made according to specific measurements done through a 3D-scan of a patient's anatomy. The attachment plate 12 provides a lateral portion 14, which is intended for abutting against the lateral side of the ramus when implanted. The mandibular prosthesis 10 provides also a head part 13 to replace the condylar head.

The attachment plate 12 provides an upper portion 16 that forms one unity with the lateral portion 14. The head part 13 comprises a mounting section 18 on the upper side 20 of the upper portion 16 of the attachment plate 12 to mount a joint head 40 for the replacement of the condylar head in the temporomandibular joint. The mounting section 18 is shaped to be morse cone like in order to mount a corresponding head part. It is also conceivable, that the mandibular prosthesis 10 forms a unity with the head part 13 (not shown).

The underside 22 of the attachment plate 12 comprises a plane support surface 24 intended for resting on a flat cutting surface 26 on the mandible, shown in FIG. 15,. Further, a medial portion 28 that is intended for abutting the medial side of the ramus and forming one unity with the upper portion 16 is shown. The lateral portion 14 comprises holes 30 arranged in a hole pattern for attachment to the lateral side of the ramus.

The alignment of the mandible to the maxilla is important in repairing a temporomandibular joint. With a mandibular prosthesis 10 an exact alignment is easily possible and all needs are beneficially facilitated. Especially the plane support surface 24 at the underside 22 of the upper portion 16 and the corresponding flat cutting surface 26 on the mandible enable an alignment and therefore a reliable function and a beneficial force transmission from the prosthesis 10 into the mandible.

The providing of the two corresponding and parallel surfaces 24, 26 make the positioning of the prosthesis 10 a safe procedure with ensured success. The cutting of the mandible and positioning is later displayed in the FIGS. 10-13, 15 and 17.

Further, the support means 14, 28 on a lateral side and a medial side of the mandibular prosthesis 10 facilitate further support and force transmission capabilities when loads from different directions are applied to the joint. A mandibular prosthesis 10 exhibiting the said characteristics can also be smaller than the previous art. A smaller prosthesis 10 does not require large incisions in the skin for implantation or a second incision positioned close to the mandibular angle, instead only a small incision is necessary in the region of the auditory canal in the area of the tragus.

The holes are preferably countersunk from the lateral side 32 of the lateral portion in order to use head locking screws 34 shown in FIG. 14, that form a thread into the countersunk part 36 of the hole 30 or in the countersunk part 36 there could be thread provided, that corresponds to the head thread 35 of the screw 34. The holes 30 are arranged inclined in various angles according to the patients anatomic situation. The holes 30 are inclined in such a way that they mostly not perpendicular to the bone surface when the prosthesis 10 is positioned onto the bone, but are inclined towards the incision made in the skin. This increases the stability of the fixation to the mandible and enables a safe way to introduce and fasten the screws 34.

In any of the following figures the embodiments of the mandibular prosthesis 10 can have different geometries, dimensions and hole patterns to the embodiment in FIG. 1, depending on the patients anatomical situation. For each replacement procedure, the mandibular prosthesis 10 and other corresponding components are custom fabricated according to the patients'anatomies.

The joint head 40 being mountable on the head part 13 is shown in FIG. 2. The joint head 40 has a mainly round ball like outer shape 42 with a flat underside 44 that opposes the upper side 20 when mounted to the mandibular prosthesis 10. Further the joint head 40 has a mounting section opening 46 on its flat underside 44, the clamped mounting is shown in the cut view in figure The joint heads 40 can be provided with various outer diameters. Additionally the depth 48 of the mounting section opening 46 reaching in to the joint head 40 can be varied in order to adjust the distance of the joint head 40 to the upper side 20 of the upper portion 16. This is necessary and beneficial to adopt the prosthesis to the various circumstances of the patients anatomy with regards to a correct positioning of the mandible.

FIG. 3 shows a cranial prosthesis 50, which is intended for the cranial side of the right side of the temporomandibular joint. The cranial prosthesis 50 has a lateral portion 52 that has been formed and is intended for abutting the lateral side of zygomatic bone of the cranium. A main body 54 is arranged below the lateral portion 52, forming one unity with the lateral portion 52.

The main body 54 has an upper portion 55 that has been formed and is intended for abutting the fossa mandibularis. Additionally a lower portion 56 with a joint pan 58 is shown, that has been formed and is intended for receiving the joint head 40 of the prosthesis 10 for the mandibular side of the temporomandibular joint.

A porous structure 60 is provided at the upper side 61 of upper portion 55, shown in FIGS. 4-8 . This porous structure 60 is intended to be osseointegrated into the cranium which secures the cranial prosthesis 50 to the cranium and ensures high forces can be derived into the bones from the joint. In that the cranial prosthesis is preferably made up of Ti6Al4V powder, the osseointegrative properties are further improved.

The lateral portion 52 of the cranial prosthesis 50 comprises holes 62 arranged in a hole pattern for attachment to the lateral side of the zygomatic arch and the fossa. The holes 62 are arranged in various angles according to the patients anatomic situation. Further the holes are preferably countersunk from the lateral side 64 of the lateral portion in order to use head locking screws 34 shown in FIG. 14 that form a thread into the countersunk part 66 of the hole 62 or in the countersunk part 66 there could be a thread provided, that corresponds to the head thread 35 of the screw 34. The holes 62 are arranged inclined in various angles according to the patients anatomic situation. The holes 62 are inclined in such a way that they mostly not perpendicular to the bone surface when the prosthesis 50 is positioned onto the bone, but are inclined towards the incision made in the skin. This increases the stability of the fixation to the fossa and zygomatic arch and enables a safe way to introduce and fasten the screws 34.

In any of the following figures the embodiments of the cranial prosthesis 50 can have different geometries, dimensions and hole patterns to the embodiment in FIG. 3, depending on the patients anatomical situation. For each replacement procedure, the cranial prosthesis 50 and other corresponding components are custom fabricated according to the patients'anatomy.

FIG. 4 shows a temporomandibular joint prosthesis 100 with the mandibular prosthesis 10, the cranial prosthesis 50 and the joint head 40 clamped onto the mounting section 18, from a temporal position. The temporomandibular joint prosthesis 100 is intended for replacing the right side of the temporomandibular joint, but it is also conceivable to provide such a prosthesis for the left side. With a left and right side of the prosthesis 100, it is possible to replace the completely temporomandibular joint. By dividing the joint replacements into the mandibular prosthesis 10, the joint head 40 and the cranial prosthesis, it can be fully customized. It is also conceivable, that the joint is only partially replaced, by using just the mandibular prosthesis 10 with the joint head 40 or the cranial prosthesis 50. Further, by dividing the prosthesis 100 into multiple components, only small incisions in the patients'skin are necessary to replace and insert the prostheses 10, 50.

In FIG. 5 a cut section of the temporomandibular joint prosthesis 100, according to FIG. 4, is shown. The planar support surface 24 on the underside of the upper portion 16 is visible from this perspective. In addition, the mounting section 18 can be seen interacting with the mounting section opening 46 and its depth 48. The distance d of the flat underside 44 to the upper side 20 can be adjusted with different depths 48. With the adjustment of the distance d, the whole height h of the prosthesis 100 and the swivel range r of the temporomandibular joint can be influenced in every direction.

FIG. 5 also shows the round shaped inside surface 68 of the joint pan 58 of the cranial prosthesis lower portion 56. The inside surface 68 is shaped corresponding to the joint head 40, but in order to facilitate the needed multi axial movement of the temporomandibular joint it can be larger than the joint head to allow for a translational movement in occipital direction or rostral direction.

The temporomandibular joint prosthesis from a medial position is shown in FIG. 6, from a rostral position in FIG. 7 and from a occipital position in FIG. 8. The porous structure 60 on the upper side 61 of the upper portion 55 is shown in these figures.

A mandibular cutting guide 200 intended for aiding the cutting of the condylar head is shown in FIG. 9. The cutting guide 200 comprises a lateral wall section 202 intended for abutment against the outside of the ramus and a guide surface 204 for guiding a cutting instrument when cutting off the condylar head along the guide surface 204. By providing a guiding surface 204, a cutting surface 26 suitable for the support surface 24 can be provided. Further a medial wall section 206 for abutment against the inside of the ramus is shown. In addition, the mandibular cutting guide 200 comprises an anterior wall section 208 and a posterior wall section 210 for abutment against the anterior and posterior side of the ramus.

It is conceivable, that the cutting guide 200 is at least partially formed of an elastically deformable material that can be bent to be placed around the condylar process. The lateral wall section 202 comprises holes 212 arranged in a hole pattern for attachment to the lateral side of the ramus. This embodiment shows two holes 212 which are arranged in a different hole pattern than the holes 30 of the mandibular prosthesis 10.

The guide surface 204 provides a cutting-start-region 214 and a cutting-end-region 216, and wherein a cutting stopper 218 is provided at the distal end of the cutting-end-region 216. The cutting stopper 218 stops the cutting tool (not shown), e.g. a saw, and prevents damage to the surrounding tissue in case the cutting tool slips off the cutting surface 204. For further safety the cutting stopper 218 provides a slot 220 that encloses the cutting-end region from three sides. Especially the muscles and tendons (not shown) are protected by the cutting stopper 218.

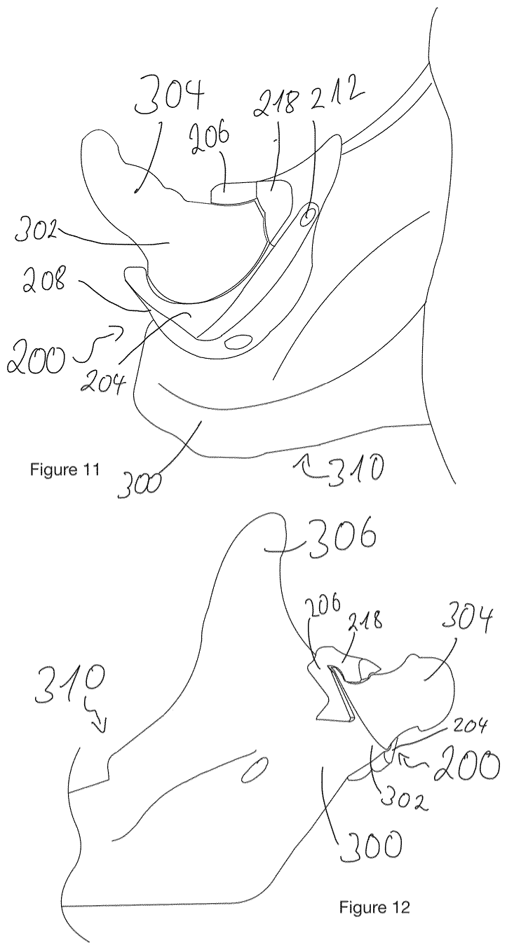

In the FIGS. 10 to 12 the mandibular cutting guide 200 is shown from different positions arranged at the ramus 300 around the condylar process 302 to cut off the condylar head 304. In FIG. 10 from a temporal position also the coronoid process 306 of the mandible 310 and the zygomatic bone 312 with the fossa 314 and the zygomatic arch 318 are shown schematically. The mandibular cutting guide 200 is fixed through the holes 212 by two screws 34 that protrude into the bone of the ramus 300. FIG. 11 shows the cutting guide 200 arranged around the condylar process 302 from a temporo-cranial position. FIG. 12 shows the cutting guide 200 arranged around the condylar process 302 from a medial position.

A one piece cutting guide 400 is shown in FIG. 13 arranged at the zygomatic arch 318 for cutting parts of the zygomatic bone in the region of the temporomandibular joint. The cutting guide 400 comprises a first part 402 and a second part 404 that form one unity.

A first part 402 a lateral wall section 406 for abutment against the outside of the zygomatic arc 318, and a planar guide surface 410 for guiding a cutting instrument when cutting off parts 412 of the zygomatic arc 318. The parts to be cut off are displayed by the dotted line 414. The guide surface 410 provides a cutting-start-region 416 and a cutting-end-region 418. A cutting stopper 420 is provided at the distal end of the cutting-end-region 418. Holes 422 are shown in the lateral portion 406 to fix the cutting guide 400 to the zygomatic arch 318 or the condylar process 302 with screws 34. With the cranial guiding surface 410 a cutting surface 423 (shown in FIG. 15) for abutting the cranial prosthesis 50 against the zygomatic arch 318 and/or the fossa 314 can be provided.

The second part 404 has a planar guide surface 424 opposing the guide surface 410 in caudal direction for guiding a cutting instrument when cutting off the condylar head 304. Through the planar guiding surface 424 a planar cutting surface 26 can be provided on the ramus 300 for the mandibular prosthesis 10.

A screw 34 for the fixation of the prostheses 10, 50 and the cutting guides 200, 400 into the bone is shown in FIG. 14. The screw 34 provides a head part 502 with the head thread 35 and a receptable 504 for a tool such as a hex drive or a torx. Further the screw 34 has an elongated body 506 with a sharp tip 508. The body 506 has a diameter D which is variable depending on the anatomy of the patient. On the body a main thread 510 is shown which is preferably a self tapping thread extending also on the tip 508. The screw 34 has an overall length L that can be varied according to the anatomy of the patient. It is also possible to have a drill guide and a drill (not shown) to drill pilot holes into the bone before inserting the screws 34, this will guide the screws 34 and make the procedure safer.

The cut surface 26 on the condylar process 302 of the ramus 300 and the cut surface 423 on the zygomatic arch 318 are shown in FIG. 15 within a schematically depicted anatomy of the temporomandibular joint region. However, providing the cut surface 423 is not mandatory to insert the cranial prosthesis 50, depending on the anatomic circumstances of the patient or if the prosthesis 50 is used at all. The cut surface 26 is produced through the use of the mandibular cutting guide 200 (FIGS. 9-12) using a cutting instrument which could be a bone saw (not displayed) and cutting along the plane provided by the guiding surface 204. Cutting starts at the cutting-start-region 214 and ends at the cutting-end-region 216 when the cutting tool is in contact with the cutting stopper 218. Alternatively, the one piece cutting guide 400 with the guiding surface 424 can be used. This ensures, that the condylar head 304 is cut off completely leaving a plane cutting surface 26 for positioning the mandibular prosthesis 10 later on. The condylar head 304 can be removed safely through the single incision in the region of the auditory canal. After removing the cut condylar head 304, the cutting guide 200 can be removed by loosening and unscrewing the screws 34 and removing the guide 200 through the single incision.

FIG. 16 shows the cranial prosthesis 50 inserted and fixed laterally to the zygomatic arch 318 by four screws 34 and abutting against the fossa 314 with the upper portion 55 of the main body 54. The porous structure 60 is preferably in contact with the cut surface 423 to enable the osseointregration of the cranial prosthesis 50. Preferably the prosthesis abuts also against the planar cut surface 423 made by using a cranial cutting guide or the one piece cutting guide 400 with the guide surface 410.

FIG. 17 shows the mandibular prosthesis 10 inserted and fixed to the mandible 310 at the ramus 300. The lateral portion 14 is abutting against the lateral side of the ramus 300 and the planar support surface 24 is resting on the flat cutting surface 26 provided at the upper side of the ramus 300. Four screws 34 through the holes 30 achieve fixing the mandibular prosthesis 10.

FIGS. 18-20 show the temporomandibular joint 100 inserted and replaced using the mandibular prosthesis 10, the cranial prosthesis 50 and the joint head 40. The mandibular prosthesis 10 is fixed to the mandible 310 and positioned according to FIG. 17. The cranial prosthesis 50 is fixed to the zygomatic arch 318 and abutting against the fossa 314 according to FIG. 16. FIG. 18 is a view from a medial-occipital position. FIG. 19 is a view from a temporal position. FIG. 20 is a view from a temporal-rostral position.

FIG. 21 shows the temporomandibular joint 100 with a view of the skull 500, wherein the left side of the temporomandibular joint is replaced using the mandibular prosthesis 10, the cranial prosthesis 50 and the joint head 40, fixed to the bone structure with screws 34.

The replacement and inserting of the temporomandibular joint 100, could be comprising the following steps:
in FIG. 22 a single access 600 through a skin incision 602 located in the area of the tragus 608 in front of the auditory canal 604 to access the temporomandibular joint and the fossa 314 is shown, the second incision 606 is just for display purposes of the mandibular prosthesis 10 and is not necessary for the procedure, a incision behind the auditory canal 604 in is also conceivable (not shown),
removal of the periosteum (not shown) on the zygomatic arch 318 in the area where the cranial prosthesis 50 is to be abutted,
removal of the periosteum on the ramus 300 in the area of the condylar process 302, where the mandibular cutting guide 200 is to be abutted,
positioning the mandibular cutting guide 200 through the single access 600 so that it abuts the mandibular notch and the medial part of the ramus and fasten the mandibular cutting guide 200 with screws 34,
cutting off the condylar process 302 through the single access 600 with a cutting instrument along the guide surface 204 of the mandibular cutting guide 200 to provide a flat cutting surface 26 at the ramus 300,
removing the screws 34 and the mandibular cutting guide 200, optionally the zygomatic bone 318 can be cut,
positioning the cranial prosthesis 50 through the access 600 so that its lateral portion 52 abuts the lateral side of zygomatic bone 318 and its upper portion 54 abuts the fossa 314,
fastening the cranial prosthesis 50 with screws 34 through the single access 600, placing a joint head 40 on the mounting section 18 of the mandibular prosthesis 10,
positioning the mandibular prosthesis 10 through the single access 60 so that its lateral portion 14 abuts the lateral side of the ramus 300 and its support surface 24 rests on the cutting surface 26 of the ramus 300,
verifying the correct match and placement between the joint head 40 of the mandibular prosthesis 10 and the joint pan 58 of the cranial prosthesis 50,
fastening the mandibular prosthesis 10 with screws 34 through the single access 600.

The verifying according to step (k) is done in case that there is an incorrect match or placement due to too large or too small clearance by removing the joint head 40 and placing and fixing a smaller or larger joint head 40 on the mounting section 18 of the mandibular prosthesis 10.

The fixing of the joint head 40 on the mounting section 18 is achieved by a hammer impact on top of the joint head 40. By shaping the mounting section 18 morse cone like the joint head 40 is clamped onto the mounting section 18.

The fastening of the cranial prosthesis 50 with screws 34 according to step (h) is done such that first the most posterior screws 34 are fastened.

The screws 34 provided for fastening the cranial prosthesis 50 and the mandibular prosthesis 10 have a diameter D from 1 mm to 4 mm, preferably of 2 mm to 3 mm and have a length L from 4 mm to 14 mm, preferably from 7 mm to 9 mm.

By providing a mandibular prosthesis 10, a cranial prosthesis 50 and a joint head 40, alone with cutting guides 200, 400, the temporomandibular joint can be replaced with a temporomandibular prosthesis 100 in a safe and easy to reproducible way. By deviding the temporomandibular prosthesis 100 into smaller components 10, 40, 50, only a small incision is necessary for the insertion. Further the prosthesis 10 can be customized at the preparation and during the procedure. By providing the cutting guides 200, 400 defined cutting surfaces 26, 423 can be made for positioning, fixing and abutting the prostheses 10, 50 on the temporomandibular joint bones.

1. A mandibular prosthesis for a mandibular side of a temporomandibular joint, comprising:
a head part to replace the condylar head, and
an attachment plate for attachment to the mandible wherein the attachment plate comprises:
a lateral portion that has been formed and is intended for abutting the lateral side of the ramus, and
an upper portion, forming one unity with the lateral portion,
wherein,
the upper portion comprises at its underside a plane support surface for resting on a flat cutting surface provided at the upper side of the ramus after removing the condylar head, the head part comprises a mounting section for mounting a joint head, wherein the mounting section is forming one unity with upper portion.

2. The mandibular prosthesis according to claim 1, comprising a medial portion that is intended for abutting the medial side of the ramus and forming one unity with the upper portion.

3. (canceled)

4. The mandibular prosthesis according to claim 1, wherein the mounting section is cylinder-shaped or cone-shaped.

5. The mandibular prosthesis according to claim 4, wherein the joint head and the mounting section are configured such that the joint head is clamped onto the mounting section.

6. The mandibular prosthesis according to claim 5, wherein the lateral portion comprises holes arranged in a hole pattern for attachment to the lateral side of the ramus

7. A mandibular cutting guide for arrangement at the ramus and at least partially around the condylar process, comprising at least
a lateral wall section for abutment against the outside of the ramus, and
a guide surface for guiding a cutting instrument when cutting off the condylar head along the guide surface, wherein the guide surface provides a cutting-start-region and a cutting-end-region, and wherein a cutting stopper is provided at the distal end of the cutting-end-region.

8. (canceled)

9. (canceled)

10. (canceled)

11. The mandibular cutting guide according to claim 7, wherein the a lateral wall section for abutment against the posteriori side of the ramus comprises holes arranged in a hole pattern for attachment to the lateral side of the ramus

12. (canceled)

13. A cranial cutting guide for arrangement at the zygomatic arch in the region of the temporomandibular joint comprising at least
a lateral wall section for abutment against the outside of the zygomatic arc, and
a planar guide surface for guiding a cutting instrument when cutting off parts of the zygomatic arc, wherein the guide surface provides a cutting-start-region and a cutting-end-region, and wherein a cutting stopper is provided at the distal end of the cutting-end-region.

14. A one piece cutting guide with a first part comprising a cranial cutting guide for arrangement at the zygomatic arch in the region of the temporomandibular joint comprising at least a lateral wall section for abutment against the outside of the zygomatic arc, and a planar guide surface for guiding a cutting instrument when cutting off parts of the zygomatic arc and a second part comprising a mandibular cutting guide for arrangement at the ramus and at least partially around the condylar process, comprising at least a lateral wall section for abutment against the outside of the ramus, and a guide surface for guiding a cutting instrument when cutting off the condylar head along the guide surface.

15. The Oone piece cutting guide according to claim 14, wherein at least one of the guide surfaces provides a cutting-start-region and a cutting-end-region, and wherein a cutting stopper is provided at the distal end of the cutting-end-region.

16. (canceled)

17. A cranial prosthesis for a cranial side of a temporomandibular joint, comprising
a lateral portion that has been formed and is intended for abutting the lateral side of zygomatic bone of the cranium,
a main body, forming one unity with the lateral portion, comprising
an upper portion that has been formed and is intended for abutting the fossa,
a lower portion with a joint pan that has been formed and is intended for receiving a joint head of a prosthesis for the mandibular side of the temporomandibular joint.

18. (canceled)

19. A temporomandibular joint comprising the mandibular prosthesis according to the claim 1 and the cranial prosthesis according to claim 17.

20. A set comprising
the mandibular prosthesis according to claim 1,
the mandibular cutting guide according to claim 7, and
joint heads of different sizes and/or with different mounting section openings to be mounted on the mounting section of the head part of the mandibular prosthesis.

21. The set according to claim 20, wherein the hole pattern of the lateral portion of the mandibular prosthesis is different than the hole pattern of the lateral wall section the mandibular cutting guide.

22. The set according to claim 20, further comprising a the cranial prosthesis according to claim 17.

23. (canceled)

24. A procedure for inserting a temporomandibular joint comprising the mandibular prosthesis according to claim 1 and the cranial prosthesis according to claim 17, comprising the following steps:
(a) providing a single access through a skin incision located in the area of the tragus in front of the auditory canal or behind the auditory canal
(b) removal of the periosteum on the zygomatic arch in the area where the cranial prosthesis is to be abutted,
(c) removal of the periosteum on the ramus in the area of the condylar process, where the mandibular cutting guide is to be abutted,
(d) positioning the mandibular cutting guide, through the single access so that it abuts the mandibular notch and the medial part of the ramus and fasten the mandibular cutting guide with screws,
(e) cutting off the condylar process through the single access with a cutting instrument along the guide surface of the mandibular cutting guide to provide a flat cutting surface at the ramus,
(f) removing the screws and the mandibular cutting guide,
(g) positioning the cranial prosthesis through the access so that its lateral portion abuts the lateral side of zygomatic bone and its upper portion abuts the fossa
(h) fastening the cranial prosthesis with screws through the single access
(i) placing a joint head on the mounting section of the mandibular prosthesis,
(j) positioning the mandibular prosthesis through the single access, so that its lateral portion abuts the lateral side of the ramus and its support surface rests on the cutting surface of the ramus,
(k) verifying the correct match and placement between the joint head of the mandibular prosthesis and the joint pan of the cranial prosthesis, and
(l) fastening the mandibular prosthesis with screws through the single access.

25. The procedure according to claim 24, wherein the verifying according to step (k) is done in case that there is an incorrect match or placement due to too large or too small clearance by removing the joint head and placing and fixing a smaller or larger joint head on the mounting section of the mandibular prosthesis.
